SEQUENCE LISTING (6 sequences)

SEQ ID NO: 1 - DNA, 18 bp, Artificial Sequence
  ZIKV-F
cgctgcccaacacaaggt

SEQ ID NO: 2 - DNA, 24 bp, Artificial Sequence
  ZIKV-R
gctccctttgccaaaaagtccaca

SEQ ID NO: 3 - DNA, 26 bp, Artificial Sequence
  ZIKV probe
accttgacaagcagtcagacactcaa

SEQ ID NO: 4 - DNA, 23 bp, Artificial Sequence
  GAPDH-F
ggtgtgaaccatgagaagtatga

SEQ ID NO: 5 - DNA, 22 bp, Artificial Sequence
  GAPDH-R
gagtccttccacgataccaaag

SEQ ID NO: 6 - DNA, 24 bp, Artificial Sequence
  GAPDH probe
agatcatcagcaatgcctcctgca

(19) United States
(12) Patent Application Publication
(10) Pub. No.: US 2020/0237698 A1
(43) Pub. Date: Jul. 30, 2020

(54) Viral Inhibitor

(71) Applicant: RUSH UNIVERSITY MEDICAL CENTER, Chicago, IL (US)

(72) Inventor: Ronen BORENSTEIN, Highland Park, IL (US)

(73) Assignee: RUSH UNIVERSITY MEDICAL CENTER, Chicago, IL (US)

(21) Appl. No.: 16/652,723

(22) PCT Filed: Oct. 3, 2018
(86) PCT No.: PCT/US2018/054102

§ 371 Date: Apr. 1, 2020

Related U.S. Application Data
(60) Provisional application No. 62/568,101, filed on Oct. 4, 2017.

(51) Int. Cl. A61K 31/201; A61K 9/00; A61P 31/12

(52) U.S. Cl. CPC A61K 31/201; A61P 31/12; A61K 9/0019; A61K 9/0014

(57) ABSTRACT

Methods of treating a viral infection in a subject are provided. The methods include administering a therapeutically effective amount of a pharmaceutical composition comprising ginkgolic acid (GA) to the subject in need thereof, where the virus comprises an enveloped virus. Further provided are specific viral infections that can be treated using the methods.

### RELATED APPLICATIONS

This application claims the benefit of U.S. Provisional Application No. 62/568,101, filed Oct. 4, 2017, which is incorporated by reference herein in their entirety.

### BACKGROUND

### 1. Field of the Invention

The present disclosure generally relates to methods useful for the treatment of a subject infected by a virus. More particularly, the disclosure relates to methods for treatment of a subject infected by a virus using therapeutically effective amount of ginkgolic acid.

### 2. Background

Extract from Ginkgo leaves is one of the most widely used herbal supplements and has become increasingly popular in recent years. Ginkgo contains two groups of active substances: flavonoid glycosides including quercetin and rutin, and terpene lactones including ginkgolides A, B, C and Ginkgolic Acid (GA). The antioxidative activity of Ginkgo compounds contributes to the protective effects seen in humans in multiple organ systems including ophthalmological, cardiovascular, pulmonary, and central nervous systems (1). GAs are 2-hydroxy-6-alkylbenzoic acids (also known as 6-alkyl salicylic acids). GAs are found in the lipid fraction of the nutshells of Ginkgo biloba and are also present in Ginkgo leaves. The commercially available compounds which are used experimentally are a simple unsaturated GA C13:0, C15:1 and C17:1, which are the main components of the nutshells and leaves. Recently, GA was reported to have activity against HIV (2), Escherichia coli and Staphylococcus aureus (3), and GA also has been reported to have antitumor effects (4). Several ways in which GA works have been suggested including by SUMOylation inhibition activity—blocking formation of the E1-SUMO intermediate (5), inhibition of fatty acid synthase (6), by Non-specific SIRT inhibition (7), and by activating protein phosphatase type-2C (8).

### BRIEF SUMMARY

Methods of treating a viral infection in a subject are provided. The methods include administering a therapeutically effective amount of a pharmaceutical composition comprising ginkgolic acid (GA) to the subject in need thereof, where the virus comprises an enveloped virus.

### FIGURE DESCRIPTION

FIGS. 1A-1E. GA inhibits HCMV in a dose-dependent manner.

FIG. 1A. Monolayers of Human foreskin fibroblasts (HFF) were infected with two clinical isolates of HCMV at constant MOI then treated with medium containing 0-10 μM of GA. Viral replication was allowed to progress for 7 days when the total number of HCMV plaques were counted, and IC50 was determined. FIGS. 1B-1E. HFF were infected with 1000 PFU of HCMV-GFP, and then treated with medium containing 0-10 μM of GA C15:1. Viral replication was allowed to progress for 7 days, when the infection was evaluated by fluorescent microscope (FIG. 1B). Inhibitory effect of 10 μM GA C15:1 compared to 16 μM GCV on HCMV-GFP infection (FIG. 1C), inhibitory effect of 10 μM GA C13:1 on HCMV-GFP infection (FIG. 1D), and inhibitory effect of 10 μM GA C17:1 on HCMV-GFP infection (FIG. 1E).

FIG. 2A. GA inhibits viral-cell fusion induced by all three classes of fusion proteins.

FIG. 2B. Inhibition of Ebola mediated cell-cell fusion by GA.

FIG. 2C. Inhibition of Ebola mediated cell-cell fusion by varied GAs.

FIG. 3A. Washing GA restores fusion.

FIG. 3B Oleic acid in solution abolishes the inhibitory effect of GA.

FIGS. 4A and 4B. GA inhibits infectivity of HSV-1 by fusion inhibition. FIG. 4A HEp2 cells. FIG. 4B 293T cells.

FIGS. 5A and 5B. GA inhibits Zika virus entry and rescues NHAs from cell death. FIG. 5A shows NHAs treated with increasing concentrations of GA and infected with ZIKV for 7 days compared to controls. FIG. 5B shows ZIKV RNA levels in infected live cells treated with increasing concentrations of GA compared to control. Experiments were performed in duplicates three independent times and the data was analyzed using T-test. * indicates p≤0.05.

FIGS. 6A-6C. FIGS. 6A and 6B illustrate that GA inhibits HSV-1 post viral infection. (6A HEp2 cells, 6B 293T cells). FIG. 6C illustrates that HCMV DNA copy number is decreased at 7 days post infection by the addition of GA to the culture medium in a dose dependent manner.

FIG. 7 illustrates that GA does not inhibit non-enveloped viruses.

### DETAILED DESCRIPTION

The embodiments disclosed below are not intended to be exhaustive or to limit the scope of the disclosure to the precise form in the following description. Rather, the embodiments are chosen and described as examples so that others skilled in the art may utilize its teachings.

Methods of treating a viral infection in a subject are provided. The methods include administering a therapeutically effective amount of a pharmaceutical composition comprising ginkgolic acid (GA) to the subject in need thereof, where the virus comprises an enveloped virus.

“Treating”, “treat”, or “treatment” within the context of the instant invention, means an alleviation of symptoms associated with a disorder or disease, or halt of further progression or worsening of those symptoms, or prevention or prophylaxis of the disease or disorder. For example, within the context of this invention, successful treatment may include an alleviation of symptoms related to viral infection. In some embodiments, the treatment may be directed to topical applications of GA. In some embodiments the treatment may be directed to acute viral infections.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition (e.g., a neutrophilic dermatosis), or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

The term “subject” or “patient” as used herein, refers to any living cellular organism that is susceptible to infection by a virus. By way of non-limiting example, cellular organisms may include mammals, including humans, fish, shrimp and other aquatic organisms, plants, mushrooms, cell lines and other organisms.

### Viruses

The viruses that can be targeted include enveloped viruses. Non-limiting examples of include viruses in the three classes of fusion proteins. Class I viruses include Zika, Ebola and Influenza hemagglutinin (HA). Class II viruses include simian foamy virus (SFV) and Western equine encephalomyelitis virus (VEEV). Class III viruses include Vesicular Stomatitis Virus (VSV) and Epstein-Barr virus (EBV). Other enveloped viruses may also be targeted, such as herpes simplex virus types 1 or 2 (HSV-1, HSV-2), Human Herpesvirus-8 (HHV-8) and other skin viruses. Additional viruses include poxviruses, influenza virus, human T cell leukemia virus (HTLV), human cytomegalovirus (HCMV), Kaposi's sarcoma-associated herpesvirus (KSHV), varicella-zoster virus (VZV), hepatitis B virus, hepatitis C virus, Marburg virus, parainfluenza virus, human respiratory syncytial virus, Hendra virus, Nipah virus, mumps virus, measles virus, Hantavirus, Bunyavirus, Rift Valley fever virus, Arenaviruses, including sin nombre virus, rabies virus, Eastern, Western and Venezuelan encephalitis viruses, West Nile virus, yellow fever virus, Dengue virus, Japanese and St. Louis encephalitis virus, coronaviruses (e.g., SARS virus), and rubellavirus.

Additional animal viruses include but are not limited to African swine fever virus (ASFV) and Foot-and-mouth disease virus (FMDV). In some embodiments, white spot syndrome virus (WSSV) or yellow head virus (YHV) that infect shrimp may be included. Rhabdovirus infectious hematopoietic necrosis virus (IHNV) that infects fish is also included. Plant viruses including but not limited to the following may be included, tobacco mosaic virus (TMV), cucumber mosaic virus (CMV), Tomato spotted wilt virus (TSWV), Tomato yellow leaf curl virus (TYLCY), Potato virus Y (PVY), Cauliflower mosaic virus (CaMV), African cassava mosaic virus (ACMV), Plum pox virus (PPV), Bromemosaic virus (BMV), Potato virus X (PVX), Citrus tristeza virus, Barley yellow dwarf virus, Potato leafroll virus and Tomato bushy stunt virus.

### Ginkgolic Acid (GA)

Ginkgolic acid is a mixture of several 2-hydroxy-6-alkylbenzoic acids in which the alkyl chain may contain Δ8 An 15:1 (I), Δ10 An 17:1 (II), or An 13:0 (III). Their structures are shown in Table 1. The three GAs C15:1, C17:1 and C13:0 may be used individually or in combination of two such as C15:1 and C17:1, C15:1 and C13:0, C17:1 and C13:0, or all three together C15:1, C17:1 and C13:1. The GA may be administered alone or in combination with one or more additional therapeutic agents. In some embodiments, the GA may be delivered topically. In other embodiments, the GA may be delivered orally.

TABLE I

| Ginkgolic Acid | Structure |
|---|---|
| C15:1 | 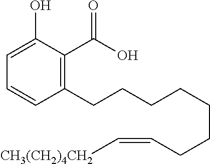 |
| C17:1 | 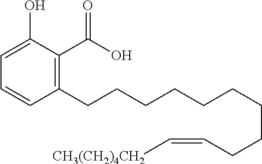 |
| C13:0 | 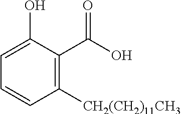 |

In certain embodiments, a pharmaceutical composition may be provided. In a related embodiment, a pharmaceutical composition of any of the compositions of the present invention and a pharmaceutically acceptable carrier or excipient of any of these compositions may be provided.

In one embodiment, a packaged treatment may be provided. The packaged treatment includes a composition of the invention packaged with instructions for using an effective amount of the composition of the invention for an intended use. In other embodiments, a use of any of the compositions for manufacture of a medicament including GA in a subject is provided.

### Pharmaceutical Compositions

The compositions described herein may be used alone or in compositions together with a pharmaceutically acceptable carrier or excipient. Pharmaceutical compositions of the present invention comprise a therapeutically effective amount of a GA and may further include one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The compounds described herein may be administered to humans and animals in dosage unit formulations containing conventional nontoxic pharmaceutically acceptable carriers, adjuvants, and vehicles as desired.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules or lipid particles, lyophilized powders, or other forms known in the art.

Compositions of the invention may be formulated for delivery as a liquid aerosol or inhalable dry powder. Liquid aerosol formulations may be nebulized predominantly into particle sizes that can be delivered to the terminal and respiratory bronchioles.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono or multi lamellar hydrated liquid crystals that are dispersed in an aqueous medium. Any non toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

Aerosolized formulations of the invention may be delivered using an aerosol forming device, such as a jet, vibrating porous plate or ultrasonic nebulizer, preferably selected to allow the formation of an aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1 5 μm. Predominantly in this application means that at least 70% but preferably more than 90% of all generated aerosol particles are within 1 5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono or diglycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

A compound described herein can be administered alone or in combination with other compounds, for a possible combination therapy being staggered or given independently of one another. Long-term therapy is equally possible as is adjuvant therapy in the con-text of other treatment strategies, as described above. Other possible treatments are therapy to maintain the patient's status after the initial treatment, or even preventive therapy, for example in patients at risk.

Effective amounts of the compounds of the invention generally include any amount sufficient to detectably an inhibition or alleviation of symptoms. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### EXAMPLES

### Material and Methods

Cells, Viruses and Ginkgolic Acid. HEp-2 cells, obtained from the American Type Culture Collection (Rockville, Md.), were grown in Dulbecco's modified Eagle medium supplemented with 5% fetal bovine serum (FBS). HEK293T/17 cells obtained from the American Type Culture Collection and maintained in Dulbecco's modified Eagle medium supplemented with 10% FBS. HSV-1(F), a limited-passage isolate, is the prototype strain used in this laboratory (9). Vero cells obtained from the American Type Culture Collection (ATCC) were cultured in complete DMEM (cDMEM) containing 10% FBS, 2 mM L-glutamine, 100 U/mL penicillin/streptomycin, and 10 mM Hepes buffer at 37° C. with 5% CO2. Fetal-derived Normal Human Astrocytes (NHAs, Lonza) were maintained in Astrocyte Growth Media (AGM, Lonza) supplemented with 0.3% FBS, 30 μl/ml, ascorbic acid, 1 μl/ml rhEGF, 30 μg/ml gentamicin, 15 μg/ml amphotericin, 2.5 μl/ml insulin, and 10 μl/ml L-glutamine. Early passages (1-4) were used in these experiments. ZIKV strain PRVABC59 obtained from ATCC was propagated in Vero cells grown in T-150 flasks by infecting at 1:50 dilution of viral stock in the absence of FBS. 6 h postinfection (hpi), supernatant was carefully removed and replaced with fresh media (cDMEM). Supernatants were collected at 72 h hpi, clarified by centrifugation at 350×g for 5 min, and filtered through a 0.45-μm surfactant-free cellulose acetate membrane. For mock infections, supernatant was collected from uninfected Vero cells and prepared by the same protocol used to make viral stocks. Virus was titered by focus assay. Briefly, infected Vero cells, 24 hpi were fixed and permeabilized using Cytofix/Cytoperm Solution Kit (BD Biosciences) according to the manufacturer's instructions and stained with a mouse monoclonal antibody (mAb) specific for flavivirus group envelope proteins (1:250; EMD Millipore; clone D1-4G2-4-15) followed by incubation with an antimouse IgG-PE (1:1000 dilution). Samples were run through an LSR II flow cytometer and data was collected with FACSDiva software (BD Biosciences) and analyzed using FlowJo Software (TreeStar).

Ginkgolic acid (GA) C13:0 (49962 Sigma-Aldrich), C15:1 (75741 Sigma-Aldrich), C17:1 (55822 Sigma-Aldrich), were purchased from Sigma Aldrich. GA was diluted in methanol or DMSO to a concentration of 50 mM. Effects of GA in all assays and models used in this study have been compared to vehicle (i.e. methanol or DMSO) treated controls. Cells or viral stocks were treated with the indicated concentration of GA.

Immunoblot Analyses: The cells were harvested at the indicated times, collected by low-speed centrifugation and rinsed in PBS, and resuspended in Ripa lysis buffer and protease inhibitors (Complete Protease Inhibitor; Roche). Approximately 60 μg of proteins per sample was subjected to further analysis. Proteins were electrophoretically separated on 8 to 10% denaturing polyacrylamide gels, electrically transferred to nitrocellulose sheets, blocked by 5% BSA in TPBS and reacted with the primary antibodies. The mouse monoclonal antibody to Us11 (Goodwin Institute for Cancer Research), the mouse monoclonal antibody to ICP8, were used in a dilution of 1:1,000. The mouse monoclonal antibody to ICP27 was used in a dilution of 1:250. The mouse monoclonal antibody to β-actin (Sigma) was used at 1:5,000 dilutions. Then, the membranes were reacted with the appropriate secondary antibody conjugated either to alkaline phosphatase or to horseradish peroxidase. Finally, protein bands were visualized with 5-bromo-4-chloro-3-indolylphosphate-nitroblue tetrazolium (Denville Scientific, Inc.) or with ECL Western blotting detection reagents (Amersham Biosciences) according to the manufacturer's instruction.

For ZIKV infection studies: NHAs were grown in 24 well plates to >80% confluency, treated with GA (0-20 μM) for 3 h and then infected with ZIKV at an MOI of 0.3. 12 hpi, supernatant was carefully removed and replaced with fresh AGM replenished with GA at 0-20 μM concentrations. 7 days postinfection, viability of the cells were assessed by Celltiter aqueous one solution cell proliferation assay (Promega) and then live cells were carefully harvested to extract RNA by RNeasy kit (Qiagen). RNA was quantified using nanodrop2000 (ThermoFisher), treated with DNasel (Sigma-Aldrich) for 15 min at RT to remove DNA contamination, and subsequently, DNasel was inactivated by heating at 70° C. for 15 min. cDNA was synthesized from 0.2-1 μg of RNA using Qscript supermix (Quanta Biosciences). Real-time PCR reactions were performed in a 20 μl solution containing 10 μl TaqMan Gene Expression Master Mix (Life Technologies), 500-nm primers and 300-nm probe. Reactions were performed in an Applied Biosystems 7900HT sequence detection system (Thermo Fisher Scientific) using SDS2.3 software. The reaction conditions were, 50° C. for 2 min, 95° C. for 10 min, followed by 45 cycles of 95° C. for 15 s and 60° C. for 1 min. Samples were run in duplicates, and no template controls were included wherever necessary. Fold change in RNA expression was calculated by relative quantification using the comparative C_{T} method with GAPDH as endogenous control. The primers and probe were designed using the PrimerQuest tool (Integrated DNA Technologies). The sequence of the primers was as follows:

(SEQ ID NO: 1)
ZIKV-F-CGCTGCCCAACACAAGGT;
(SEQ ID NO: 2)
ZIKV-R-, GCTCCCTTTGCCAAAAAGTCCACA
and
(SEQ ID NO: 3)
ZIKV-probe, 5′/56-FAM/ACCITGACA/ZEN/
AGCAGTCAGACACTCAA/3IABkFQ;
and
(SEQ ID NO: 4)
human specific GAPDH-F-GGTGTGAACCATGAGAAGTATGA;
(SEQ ID NO: 5)
GAPDH-R-GAGTCCTTCCACGATACCAAAG;
and
(SEQ ID NO: 6)
GAPDH-probe, 5′/56-FAM/AGATCATCA/ZEN/
GCAATGCCTCCTGCA/3IABkFQ.

### Results

### GA Inhibits HCMV Infectivity

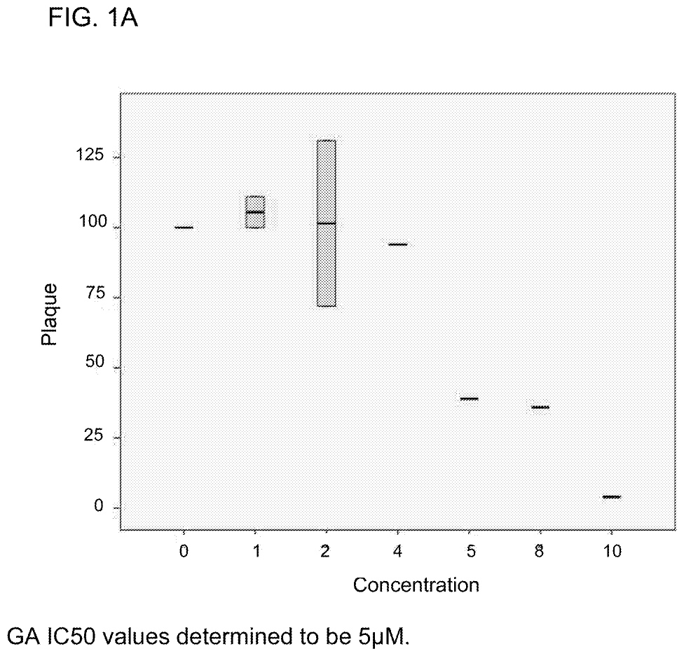

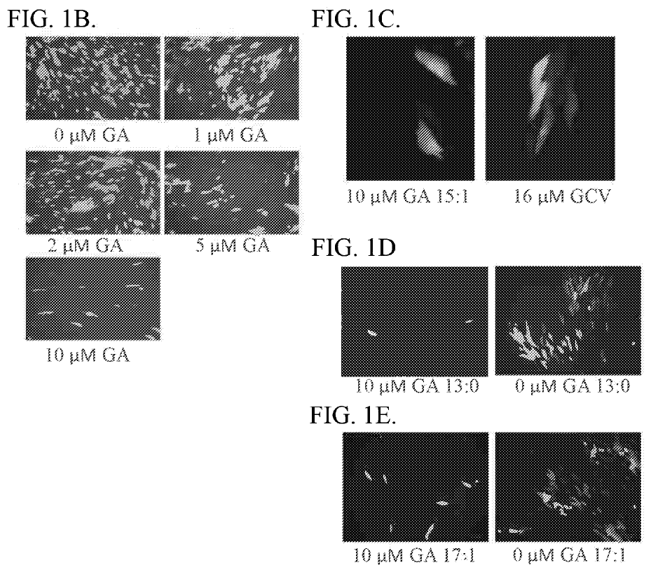

To assess the effect of GA on HCMV infection, dilutions of 1 μM to 20 μM were made (FIG. 1A). Monolayers of Human foreskin fibroblasts (HFF) were infected with two clinical isolates of HCMV, then treated with medium containing 0-10 μM of GA C15:1. Viral replication was allowed to progress for 7 days, and infectivity was monitored by plaquing efficiency. As shown in FIG. 1A, GA C15:1 inhibited HCMV infectivity.

To access the effect of GA on plaque formation, HCMV-GFP virus was used (FIG. 1B). As expected by the determined IC50, at 5 and at 10 μM C15:1, the effect was exhibited. Furthermore, at 10 μM C15:1, only single cells were infected, indicating that there was no cell-to-cell viral spread, implying that the GA is working on the viral membrane. To verify these results we compared the inhibition of infection by GA C15:1 to that of Ganciclovir (GCV), the most known potent drug against HCMV. The results showed that at 16 μM of GCV, although there was a very strong inhibition of the virus, there were still plaques visible indicating a cell-to-cell spread. Whereas at the 10 μM GA C15:1 treated infections, there were only separate infected cells, supporting our estimation that the main mechanism which GA inhibits HCMV is by preventing viral fusion (FIG. 1C). We next tested GA C17:1 and GA C13:0. Both GA compounds had a strong inhibitory effect on HCMV infection, similar to GA C15:1 (FIG. 1D). All of the GA forms did not show toxicity at the active concentration (Not shown).

### GA Inhibits Cell Fusion Induced by All Three Classes of Fusion Proteins

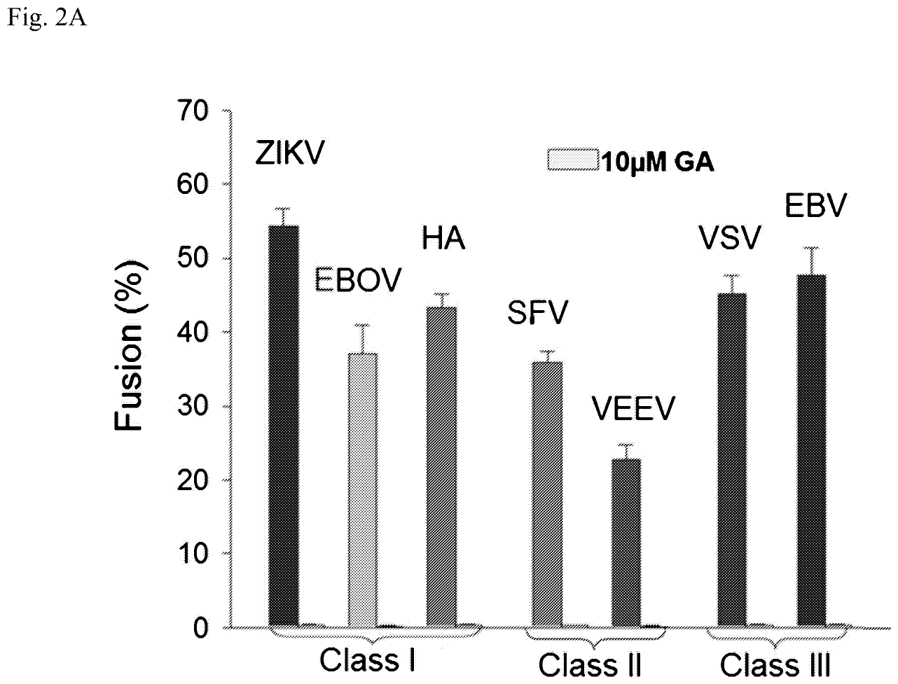

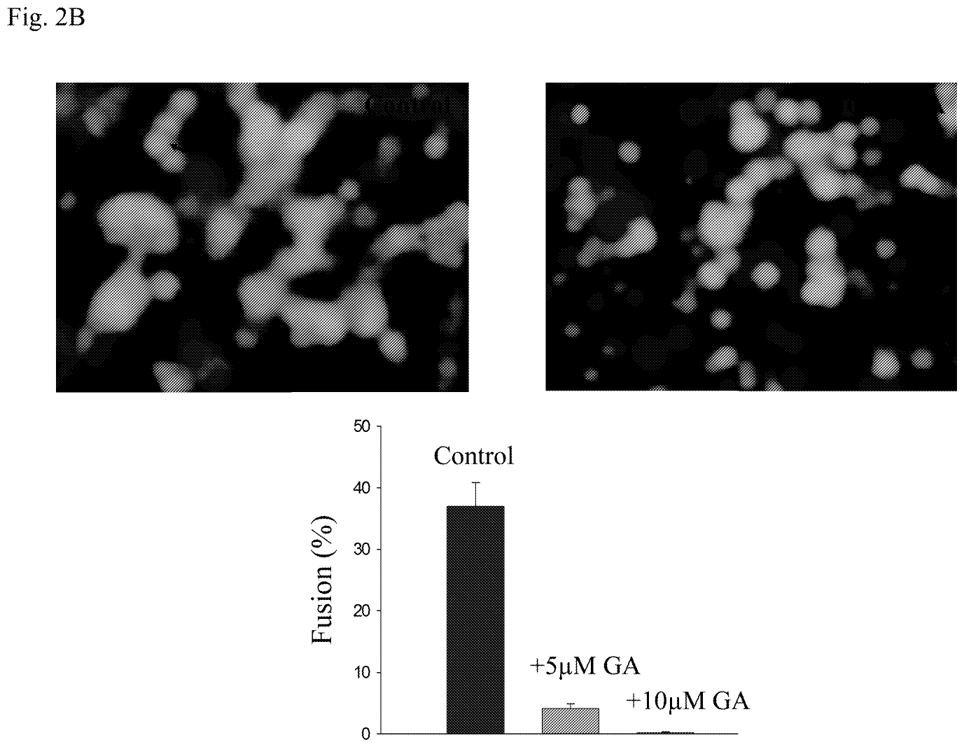

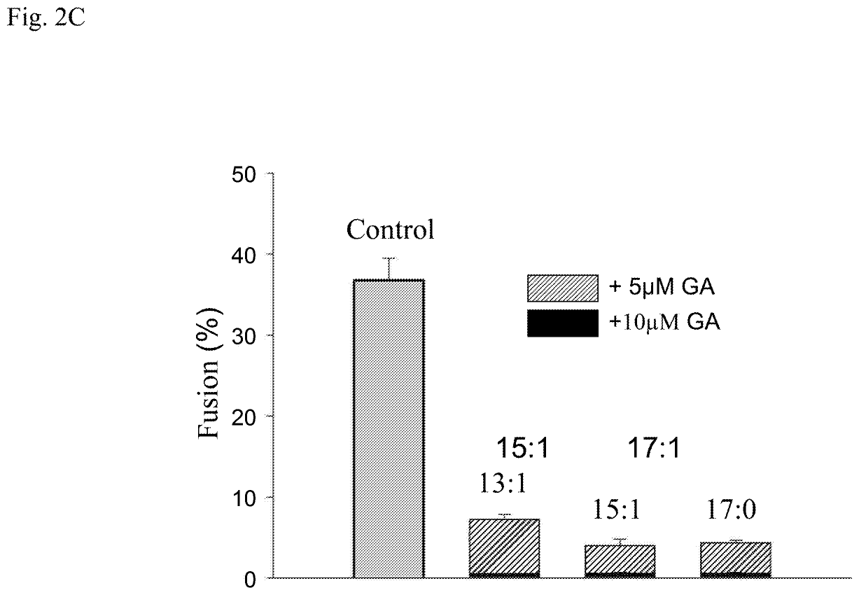

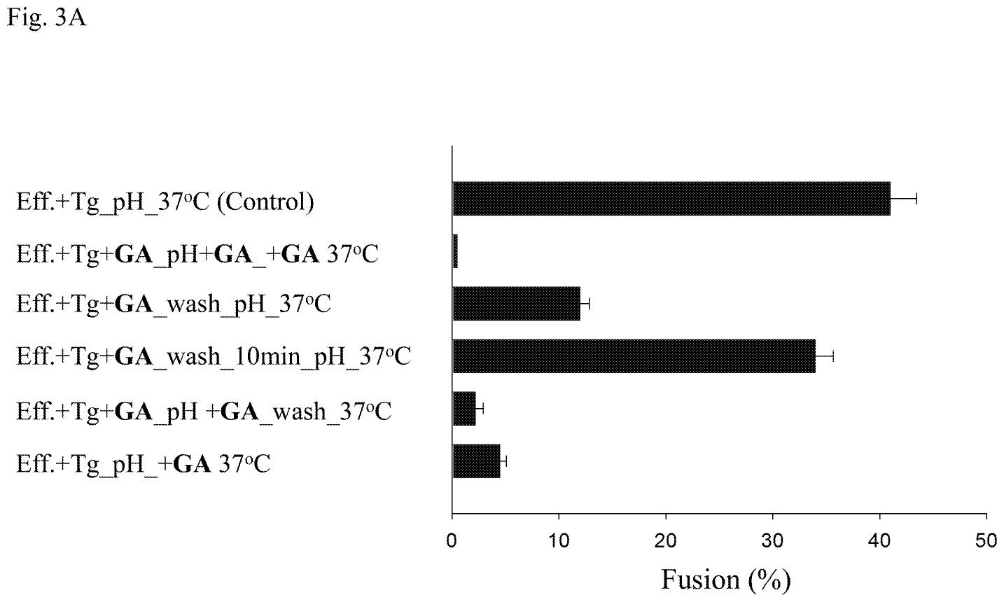

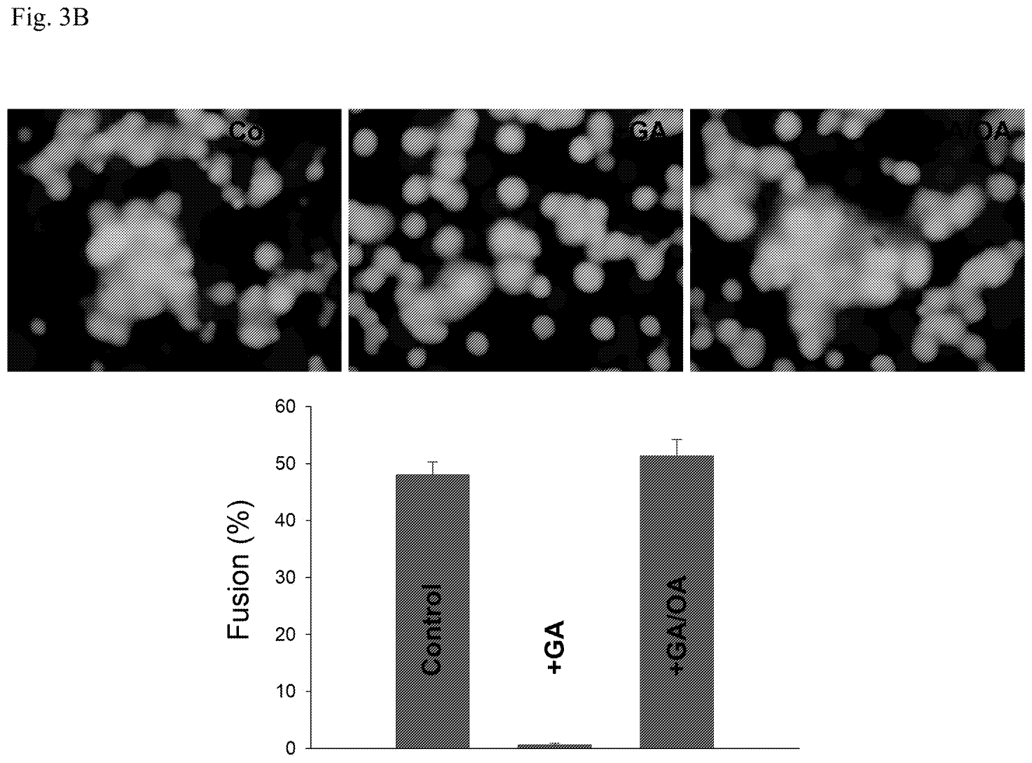

To assess the activity of GA on viral-cell fusion, we utilize fluorescent dye spread assays to monitor cell-cell fusion. Effector COS7 cells transfected to express all three classes of fusion proteins, including Zika, EBOV, influenza A virus (IAV), simian foamy virus (SFV), Western equine encephalomyelitis virus (VEEV), Vesicular Stomatitis Virus (VSV) and Epstein-Barr Virus (EBV). The COS7 cells were bound to 293T target cells that were either unlabeled or, for purposes of microscopic identification, loaded with the aqueous dye CMAC (blue). All three classes of fusion proteins were 100% blocked in the presence of 10 μM GA 13:0, GA 15:1 and GA 17:1 (FIG. 2A illustrates C15:1 inhibition). FIG. 2B shows inhibition of Ebola mediated cell-cell fusion by GA at 5 μM and 10 μM GA C15:1. FIG. 2C shows inhibition of Ebola mediated cell-cell fusion by GA C13:1, GA C15:1 and GA C17:0 at two different concentrations each. When GA was washed, fusion was restored, indicating that GA interferes with fusion in a non-covalent way (FIG. 3A). The cone shaped lipid oleic acid (OA) has a negative spontaneous curvature, which favors hemifusion when present in the outer bilayer. The addition of OA together with GA abolished the inhibitory effect of GA (FIG. 3B), indicating that GA has a positive spontaneous curvature.

### GA Inhibits Infectivity of Different Types of Enveloped Viruses by Fusion Inhibition

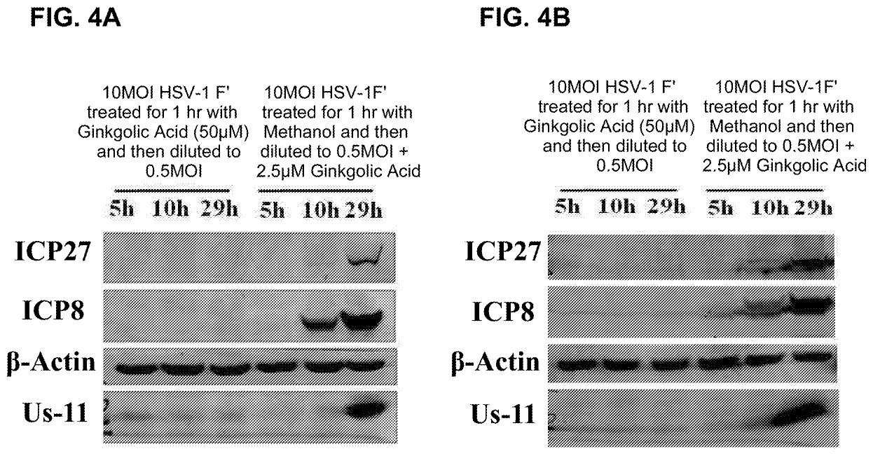

We next tested GA C15:1 on HSV-1, a strong and fast infectious DNA virus, and on the Zika RNA virus that have neither glycoprotein conservation nor common receptors with HCMV and HSV-1. To test whether GA inhibits HSV-1 by fusion inhibition, we designed an experiment to test the direct effect of GA on the virus. For this experiment, we used HEp2 and 293T cells. 10 MOI HSV-1 F′ were treated for 1 hour with GA (50 μM, C15:1) or with vehicle in serum free DMEM and then diluted to 0.5 MOI in 199V medium in a final concentration of 2.5 μM GA C15:1. As a control, the virus that was treated with the vehicle was supplemented also with 2.5 μM GA C15:1. 6 well cultures of HEp2 (FIG. 4A) or 293T (FIG. 4B) were then infected and 5, 10 and 29 hours post-infection cells were collected and fraction of total cell lysate was subject to western blotting (WB). To test the viral infection, HSV-1 immediate early (ICP27), early (ICP8) and late (Us11) proteins were detected by WB (FIGS. 4A & B). The results showed that in the treated 10 MOI HSV-1 F′ stock there was a complete inhibition of the virus, implying there was fusion inhibition.

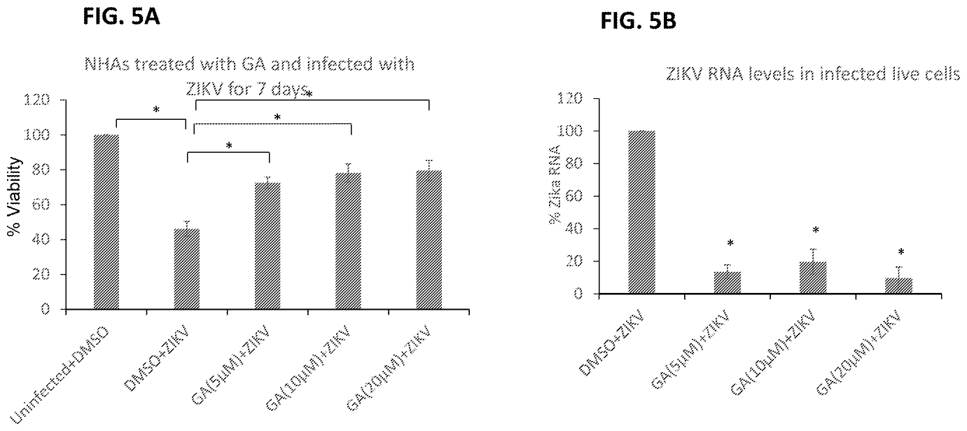

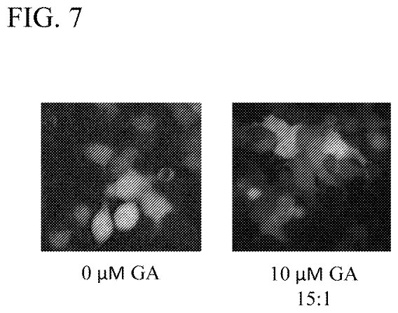

To test the effect of GA C15:1 on Zika virus infectivity, Normal Human Astrocytes (NHAs) were grown to 90% confluency in a 24 well plate, treated with GA (0-20 mM, C15:1) or DMSO for 3 hours without serum and then infected with ZIKV (strain PRVABC59) at a MOI of 0.25. The next day, supernatant was replaced with fresh media containing drug or DMSO and cells were incubated at 37 C, 5% CO2. At day 7, cells were treated with MTS for viability assay, and then harvested to extract total RNA for quantification of Zika viral RNA by Taqman based real-time PCR. The results showed 70% to 80% viability at 5 μM to 20 μM GA C15:1, compared to less than 40% viability at the vehicle treated cells. Furthermore, the quantification of the Zika viral RNA, showed 80-90% decrease in RNA levels at 5 μM to 20 μM GA C 15:1 (FIGS. 5A & B). We concluded that GA inhibits Zika virus entry and rescues NHAs from cell death. Furthermore, the targets of GA are therefore conserved among viruses with no conserved glycoproteins, and they use different receptors and fuse to different cell membranes. To determine whether GA is active against nonenveloped viruses, we also tested its activity on adenovirus nonenveloped viruses that are internalized by endocytosis. Monolayers of HEp2 cells were incubated for 1 hour with medium containing 10 μM of GA C13:0, C15:1, C17:1 and DMSO vehicle. The cells then were infected with human Adenovirus Type 5 (dE1/E3) containing GFP (Ad-GFP), at 20 MOI in a medium containing 10 μM GA for 24 hours. Infection was evaluated by fluorescent microscope. As shown in FIG. 7, GA did not inhibit the nonenveloped Adenovirus.

### GA Inhibits Infectivity of Different Types of Enveloped Viruses by Inhibition of Viral DNA and Protein Synthesis

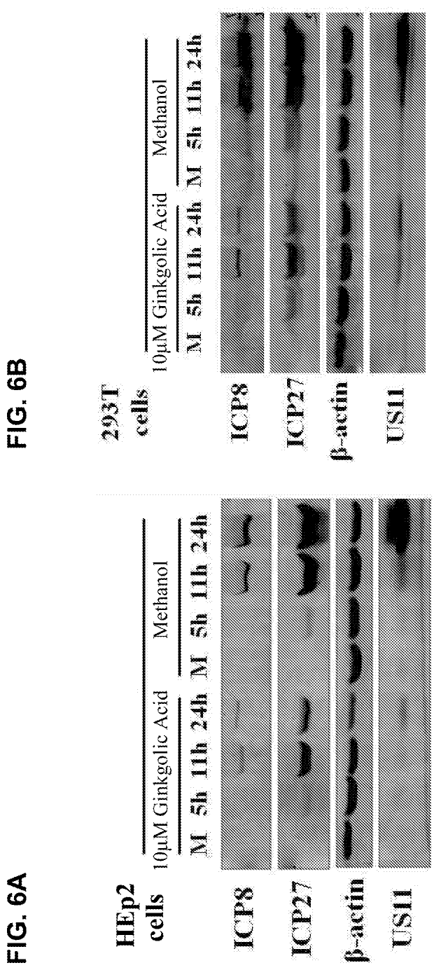

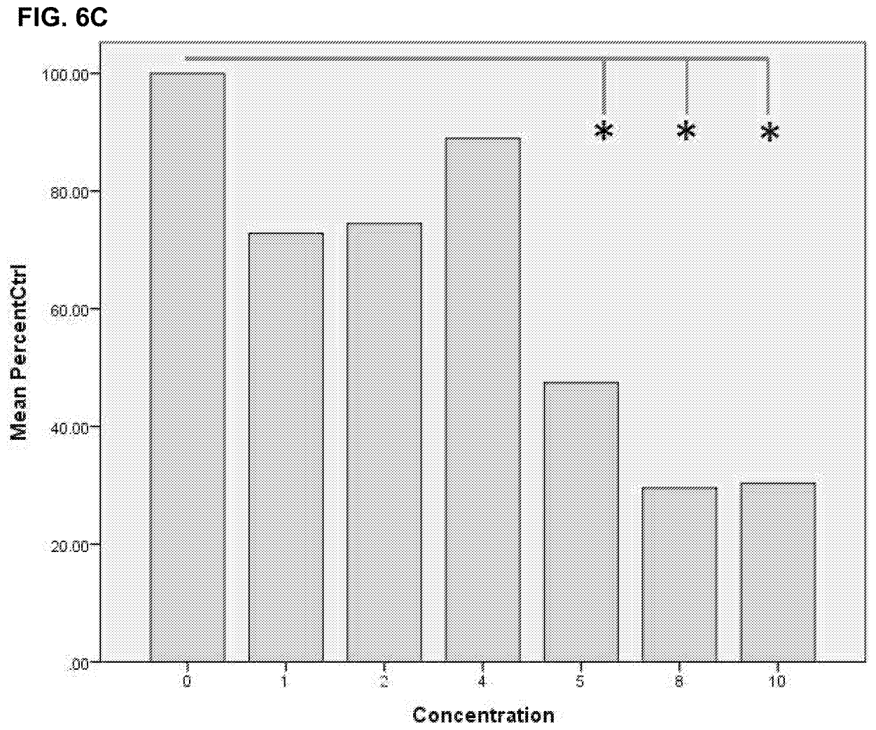

To test whether GA works in a secondary mechanism, we designed an experiment to evaluate the effect of 10 μM GA C15:1 on already infected cells. Untreated HEp2 (FIG. 6A) and 293T (FIG. 6B) cells were infected with HSV-1 F′ 1 MOI for 2 hours, allowing the virus to internalize into the cells, then washed with 199V medium and supplemented with 10 μM GA C15:1 or vehicle. The infection was evaluated by WB detection of HSV-1 proteins. Even though the infection had begun, the addition of 10 μM GA C15:1 to the infected HEp2 and 293T started to work immediately, inhibiting the virus from that point (FIGS. 6A, 6B). These results may imply that there is a secondary inhibition mechanism inhibiting viral protein synthesis.

To test whether there is effect on viral DNA synthesis, we used HCMV infections followed by RT-PCR. HFF were infected with HCMV for 3 hours, then washed and incubated with different concentrations of GA or vehicle. DNA was extracted from 4 pooled wells of HFF at 7 dpi, and then analyzed by qPCR targeting the viral polymerase gene. DNA copies were reduced in a dose dependent manner by the addition of GA to culture medium (FIG. 6C). The results showed high correlation between GA concentration and the drop in viral DNA. Although we treated the infected cells with GA only after the virus internalized, there was still a strong inhibiting effect on viral DNA synthesis resulting in about 80% reduction.

### Discussion

The GAs C13:0, C15:1 and C17:1 are commercially available compounds of Ginkgo leaves. To assess the effect of GA on infectious viruses, we developed GA dose experiments. The cells were treated with different GA concentrations ranging between 1 μM to 20 μM. We demonstrated a dose dependent effect of GA on HCMV, HSV-1 and Zika viruses. The effect of GA was tested in several cell types including HEp2 (adherent human epithelial carcinoma), 293T (adherent human embryonic kidney), Human foreskin fibroblasts (HFF) and Normal Human Astrocytes (NHAs), with no toxicity at the active inhibitory range. GA was shown to have a viral inhibitory effect in all the tested cells.

In conclusion, we showed a very strong inhibitory effect of GA on the fusion of enveloped viruses. These molecules are active against a variety of enveloped viruses, including important pathogens such as EBOV, ZIKA, HSV-1, HCMV, EBV and Influenza A. Furthermore, we found that GA inhibits viral DNA and protein synthesis by a secondary mechanism.

Thus, in light of the strong effect of GA on viral infection, even after the infection begins, GA may be used to treat acute infections (eg. Ebola, Zika), and also topically for the successful treatment of active lesions (HSV-1, HSV-2, HHV-8 and all enveloped viruses associated with the skin).

### In Vivo Assays With GA

### Herpes Simplex Virus (HSV)

Mouse models of herpes simplex virus type 1 or type 2 (HSV-1 or HSV-2) will be employed to assess the antiviral activity of GA (C15:1, C17:1 and C13:0, individually and/or combinations thereof) in vivo. Mice will be inoculated by various routes with an appropriate multiplicity of infection of HSV (e.g., 10⁵ pfu of HSV-1 or 4×10⁴ pfu of HSV-2) followed by administration of GA and placebo. For i.p. inoculation, HSV-1 replicates in the gut, liver, and spleen and spreads to the CNS. For i.n. inoculation, HSV-1 replicates in the nasaopharynx and spreads to the CNS. Any appropriate route of administration (e.g., oral, topical, systemic, nasal), frequency and dose of administration can be tested to determine the optimal dosages and treatment regimens using GA, optionally in combination with other therapies.

In a mouse model of HSV-2 genital disease, intravaginal inoculation of female Swiss Webster mice with HSV-1 or HSV-2 will be carried out, and vaginal swabs will be obtained to evaluate the effect of therapy on viral replication (See, e.g., Crute et al., Nature Medicine, 2002, 8:386-391). For example, viral titers by plaque assays are determined from the vaginal swabs. A mouse model of HSV-1 using SKH-1 mice, a strain of immunocompetent hairless mice, to study cutaneous lesions may also be used (See, e.g., Crute et al., Id. and Bolger et al., Antiviral Res., 1997, 35:157-165). Guinea pig models of HSV have also been described, See, e.g., Chen et al., Virol. J, 2004 Nov. 23, 1:11. Statistical analysis will be carried out to calculate significance (e.g., a P value of 0.05 or less).

### HCMV

Mouse models of infection with murine CMV (MCMV) will be used to assay the antiviral activity of GA (C15:1, C17:1 and C13:0, individually and/or combinations thereof) in vivo since HCMV does not generally infect laboratory animals. For example, a MCMV mouse model with BALB/c mice can be used to assay the antiviral activities of GAs in vivo when administered to infected mice (See, e.g., Kern et al., Antimicrob. Agents Chemother., 2004, 48:4745-4753). Tissue homogenates isolated from infected mice treated or untreated with GA will be tested using standard plaque assays with mouse embryonic fibroblasts (MEFs). Statistical analysis will be done to calculate significance (e.g., a P value of 0.05 or less).

Alternatively, human tissue (i.e., retinal tissue or fetal thymus and liver tissue) will be implanted into SCID mice, and the mice will subsequently be infected with HCMV, preferably at the site of the tissue graft (See, e.g., Kern et al., Antimicrob. Agents Chemother., 2004, 48:4745-4753). The pfu of HCMV used for inoculation can vary depending on the experiment and virus strain. Any appropriate routes of administration (e.g., oral, topical, systemic, nasal), frequency and dose of administration will be tested to determine the optimal dosages and treatment regimens using GAs, optionally in combination with other therapies. Implant tissue homogenates isolated from infected mice treated or untreated with GAs at various time points will be tested using standard plaque assays with human foreskin fibroblasts (HFFs). Statistical analysis will be done to calculate significance (i.e., a P value of 0.05 or less).

Treatment of African swine fever virus (ASFV) using GAs including C15:1, C13:0 and C17:1 and combinations thereof will be evaluated. Example protocols may be found in Hakobyan et al., Arch Virol (2016) 161:3445-3453, which is incorporated by reference herein.

Treatment of Foot-and-mouth disease virus (FMDV) in cloven-hoofed animals such as cattle, swine, and sheep using GA will be evaluated. Treatment using GAs including C15:1, C13:0 and C17:1 and combinations thereof will be evaluated. Example protocols may be found in Zhao et al., J Med Virol. 2017; 89:2041-2046, which is incorporated by reference herein.

Treatment of white spot syndrome virus (WSSV) or yellow head virus (YHV) with GA in shrimp cultures will be evaluated. Treatment using GAs including C15:1, C13:0 and C17:1 and combinations thereof will be evaluated. Example protocols may be found in Maikaeo et al., Dis Aquat Org 115: 157-164, 2015, which is incorporated by reference herein.

Fish rhabdovirus infectious hematopoietic necrosis virus (IHNV) will be used as a model to study treatment of aquatic enveloped virus diseases using GA. GAs including C15:1, C13:0 and C17:1 and combinations thereof will be evaluated. Example protocols may be found in Balmer B F et al., J Virol., 2016, 91:e02181-16, which is incorporated by reference herein.

Treatment of plant viruses such as tobacco mosaic virus (TMV) and cucumber mosaic virus (CMV) using GAs including C15:1, C13:0 and C17:1 and combinations thereof will be evaluated. Treatment of additional plant viruses using GAs include the following but are not limited thereto, Tomato spotted wilt virus (TSWV), Tomato yellow leaf curl virus (TYLCY), Potato virus Y (PVY), Cauliflower mosaic virus (CaMV), African cassava mosaic virus (ACMV), Plum pox virus (PPV), Bromemosaic virus (BMV), Potato virus X (PVX), Citrus tristeza virus, Barley yellow dwarf virus, Potato leafroll virus and Tomato bushy stunt virus. Example protocols may be found in Yu et al., Molecules 2017, 22, 658, which is incorporated by reference herein.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

### REFERENCES

1. Zhou W, Chai H, Lin P H et al. Clinical use and molecular mechanisms of action of extract of Ginkgo biloba leaves in cardiovascular diseases. Cardiovasc Drug Rev, 2004; 22: 309-19.

2. Lü J M, Yan S, Jamaluddin S, Weakley S M, Liang Z, Siwak E B, Yao Q, Chen C. Ginkgolic acid inhibits HIV protease activity and HIV infection in vitro. Med Sci Monit. 2012 Aug. 18 (8):BR293-298.

3. Lee J H, Kim Y G, Ryu S Y, Cho M H, Lee J. Ginkgolic acids and Ginkgo biloba extract inhibit Escherichia coli 0157:H7 and Staphylococcus aureus biofilm formation. Int J Food Microbiol. 2014 Mar. 17; 174:47-55.

4. Antitumor effects of ginkgolic acid in human cancer cell occur via cell cycle arrest and decrease the Bcl-2/Bax ratio to induce apoptosis. Zhou C, Li X, Du W, Feng Y, Kong X, Li Y, Xiao L, Zhang P. Chemotherapy. 2010; 56 (5):393-402.

5. Fukuda I, Ito A, Hirai G, Nishimura S, Kawasaki H, Saitoh H, Kimura K, Sodeoka M, Yoshida M. Ginkgolic acid inhibits protein SUMOylation by blocking formation of the E1-SUMO intermediate. Chem Biol. 2009 Feb. 27; 16 (2):133-40.

6. Oh J, Hwang I H, Hong C E, Lyu S Y, Na M. Inhibition of fatty acid synthase by ginkgolic acids from the leaves of Ginkgo biloba and their cytotoxic activity. J Enzyme Inhib Med Chem. 2013 Jun. 28 (3):565-8.

7. Ryckewaert L, Sacconnay L, Carrupt P A, Nurisso A, Simões-Pires C. Non-specific SIRT inhibition as a mechanism for the cytotoxicity of ginkgolic acids and urushiols. Toxicol Lett. 2014 Sep. 2; 229 (2):374-80.

8. Ahlemeyer B, Selke D, Schaper C, Klumpp S, Krieglstein J. Ginkgolic acids induce neuronal death and activate protein phosphatase type-2C. Eur J Pharmacol. 2001 Oct. 26; 430 (1):1-7.

9. Ejercito, P. M., E. D. Kieff, and B. Roizman. 1968. Characterization of herpes simplex virus strains differing in their effects on social behaviour of infected cells. J. Gen. Virol. 2:357-364.

10. Baek S H, Lee J H, Kim C, Ko J H, Ryu S H, Lee S G, Yang W M, Um J Y, Chinnathambi A, Alharbi S A, Sethi G, Ahn K S. Ginkgolic Acid C 17:1, Derived from Ginkgo biloba Leaves, Suppresses Constitutive and Inducible STAT3 Activation through Induction of PTEN and SHP-1 Tyrosine Phosphatase. Molecules. 2017 Feb. 13; 22 (2).

11. Mango D, Weisz F, Nisticò R. Front Pharmacol. 2016 Oct. 26; 7:401. Ginkgolic Acid Protects against Aβ-Induced Synaptic Dysfunction in the Hippocampus.

12. Hua Z, Wu C, Fan G, Tang Z, Cao F. The antibacterial activity and mechanism of ginkgolic acid C15:1. BMC Biotechnol. 2017 Jan. 14; 17 (1):5.

1. A method of treating a viral infection in a subject comprising:
administering a therapeutically effective amount of a pharmaceutical composition comprising ginkgolic acid (GA) to the subject in need thereof,
wherein the virus comprises an enveloped virus that is selected from Zika, Ebola, Influenza hemagglutinin (HA), simian foamy virus (SFV), Western equine encephalomyelitis virus (VEEV), Vesicular Stomatitis Virus (VSV), Epstein-Barr virus (EBV), herpes simplex virus-1 (HSV-1) and human cytomegalovirus (HCMV).

2. The method according to claim 1, wherein the GA is selected from GA C15:1, GA C17:1, GA C13:0 and combinations thereof.

3. The method according to claim 1, wherein the pharmaceutical composition is administered topically.

4. The method according to claim 1, wherein the pharmaceutical composition is administered intravenously.

5. The method according to claim 1, wherein the pharmaceutical composition is formulated together with a pharmaceutically acceptable carrier or excipient.

6. The method according to claim 3, wherein the virus is selected from HSV-1 and HCMV.

7. The method according to claim 4, wherein the virus is selected from Ebola and Zika.

8. The method according to claim 1, wherein the GA is GA C15:1.

9. The method according to claim 1, wherein the GA is GA C17:1.

10. The method according to claim 1, wherein the GA is GA C13:0.

11. The method according to claim 1, wherein the GA is GA C15:1, GA C17:1, and GA C13:0.
